(19) United States
(12) Patent Application Publication
(10) Pub. No.: US 2013/0204269 A1
(43) Pub. Date: Aug. 8, 2013

(54) Intraocular Lens Injector with Vibration

(71) Applicant: Bausch & Lomb Incorporated, Rochester, NY (US)

(72) Inventor: Joel Pynson, Toulouse (FR)

(73) Assignee: Bausch & Lomb Incorporated, Rochester, NY (US)

(21) Appl. No.: 13/756,602

(22) Filed: Feb. 1, 2013

Related U.S. Application Data
(60) Provisional application No. 61/594,096, filed on Feb. 2, 2012.

(51) Int. Cl. A61F 2/16

(52) U.S. Cl. CPC A61F 2/167
USPC 606/107

(57) ABSTRACT

An intraocular lens injector, comprising an injector body having a lumen wall defining a lumen, a plunger configured and arranged to move an IOL through the lumen, and a vibration source configured and arrange to vibrate at least one of the injector body and the plunger.

### CROSS REFERENCE

This application claims the benefit of Provisional Patent Application No.: 61/594096, filed on Feb. 2, 2012 which is incorporated by reference herein.

### FIELD OF INVENTION

The present invention relates to intraocular lens (IOL) injectors, and more particularly to IOL injectors with vibration.

### BACKGROUND OF THE INVENTION

IOLs are artificial lenses used to replace or supplement the natural crystalline lenses of patients' when their natural lenses are diseased or otherwise impaired. IOLs may be placed in either the posterior chamber or the anterior chamber of an eye. IOLs come in a variety of configurations and materials.

Various instruments and methods for implanting such IOLs in an eye are known. Typically, an incision is made in a patient's cornea and an IOL is inserted into the eye through the incision. In one technique, a surgeon uses surgical forceps to grasp the IOL and insert it through the incision into the eye. While this technique is still practiced today, more and more surgeons are using IOL injectors, which offer advantages such as affording a surgeon more control when inserting an IOL into an eye and petmitting insertion of IOLs through smaller incisions.

In order for an IOL to fit through a small incision, it is typically folded and/or compressed by passing the IOL through an injector having a funnel-shaped lumen. After the IOL exits the distal end of the lumen and enters the eye, it assumes its original unfolded/uncompressed shape.

Relatively small incision sizes are preferred over relatively large incisions since smaller incisions have been attributed with reduced post-surgical healing time and reduced complications such as induced astigmatism. In many instances, it is desirable that the incision size be less than 2.0 mm or even less than 1.5 mm. However, the amount of force applied to the lens to deliver it through such an incision increases as the incision size decreases. While various coatings and lubricants have been applied to the lumen walls to decrease the force necessary to deliver an IOL, there remains a need for further techniques to reduce the force applied to the IOL and thereby decrease the likelihood of damaging the IOL during delivery to an eye.

### SUMMARY

Aspects of the present invention are directed to an intraocular lens injector comprising an injector body having a lumen wall defining a lumen terminating at an open end configured for delivery of an IOL into an eye, a plunger configured and arranged to move an IOL through the lumen, and a vibration source configured and arrange to vibrate at least one of the injector body and the plunger tip. To achieve vibration of the at least one of the injector body and the plunger tip, a source of vibration (e.g., a motorized vibration apparatus) may be coupled, for example, to the injector body, the plunger or other component of the injector.

In some embodiments, the lumen wall is sized and shaped to fold or compress the IOL as it moves through the lumen.

The plunger may include threading to facilitate longitudinally through the lumen. The injector may be motorized to achieve longitudinal motion of the plunger through the lumen.

The vibration source may be directly connected to injector body. For example, the vibration source may be connected to an exterior surface of the injector.

In some embodiments, the power source for vibration source is attached to the injector so as to be supported by the injector.

In some embodiments, the power for the vibration source is provided by an external apparatus.

The term “vibration” as used herein refers to an oscillatory movement about an equilibrium. Vibration may be regular or random.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic illustration of an example of an injector according to aspects of the present invention; and

FIG. 2 is a schematic illustration of an example of an injector according to aspects of the present invention in which the vibration source is connected to an exterior surface of the injector body.

### DETAILED DESCRIPTION

Aspects of the present invention are directed towards an IOL injector comprising an injector body having a lumen extending therethrough, a plunger adapted to move an IOL (also referred to herein using the term “lens”) through the lumen and through an open end in the injector, and a source of vibration. The source of vibration is operatively coupled to at least one of the injector body and the plunger so as to vibrate at least one of the injector body and the plunger thereby decreasing the force needed to be applied to plunger to move the IOL through the lumen into an eye.

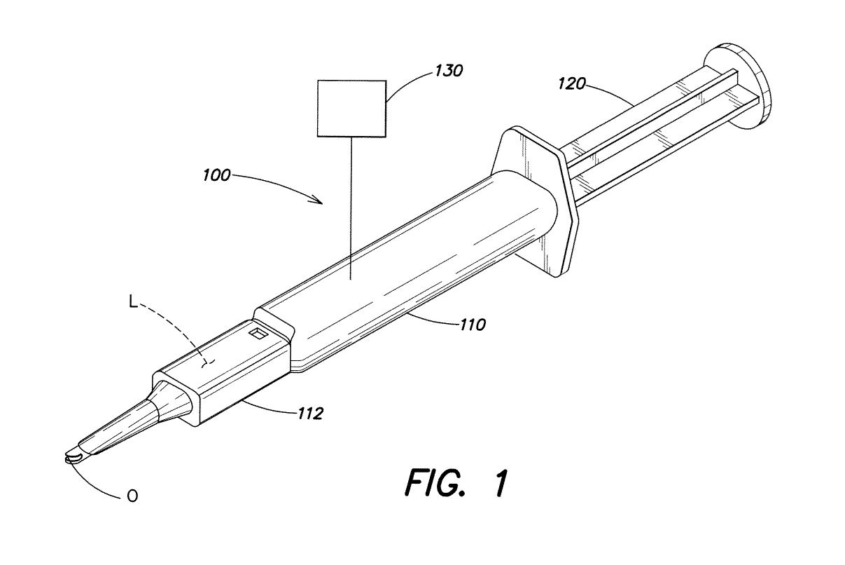

FIG. 1 illustrates an example of an intraocular lens injector 100 according to aspects of the present invention. The injector comprises an injector body 110, a plunger 120 and a source of vibration 130.

Injector body 110 has a lumen wall 112 defining a lumen L, and an opening O for delivering the IOL into an eye. The lumen is configured and arranged to permit controlled movement of an IOL into an eye. The lumen is sized and shaped to fold, compress and/or otherwise form the lens for delivery into an IOL when the IOL exits the injector through an opening O at the end of the lumen. Typically, the lumen wall is tapered such the lens is compressed as the lens progresses from the proximal end to the distal end of the lumen. It will be appreciated that, in such embodiments, the amount of force needed to actuate the plunger increases as the lens proceeds through portions of the lumen having a reduced cross sectional area; and application of additional force results in an increased likelihood of damage to the IOL. It will also be appreciated that the injector body may have any suitable configuration and can be divided into multiple portions. For example, the body may comprise a disposable cartridge portion. According to aspects of the present invention, by providing vibration to one or more portions of the injector, the IOL and/or the injector body are caused to vibrate, thereby reducing the amount of force needed to actuate the plunger.

The plunger is configured and arranged to move through the lumen and may have any suitable configuration. The plunger may include threading configured to interact with threading on the injector body so that the plunger can be rotated to cause movement down the lumen; alternatively the plunger may simply slide longitudinally through the lumen. The plunger may have any suitable tip configuration to contact the IOL. For example, the tip may have a curved or forked shape, and may be covered with a relatively soft material, the material being softer than the plunger shaft. For example, the tip may comprise a silicone, a thermoplastic polyurethane, a styrene butadiene styrene, a styrene isoprene styrene, a styrene ethylene/butylene styrene, a polyether block amide, a styrenic thermoplastic or a fluoroelastomer (e.g. sold under the brand name Viton). The plunger may be actuated manually or using a motorized assistance.

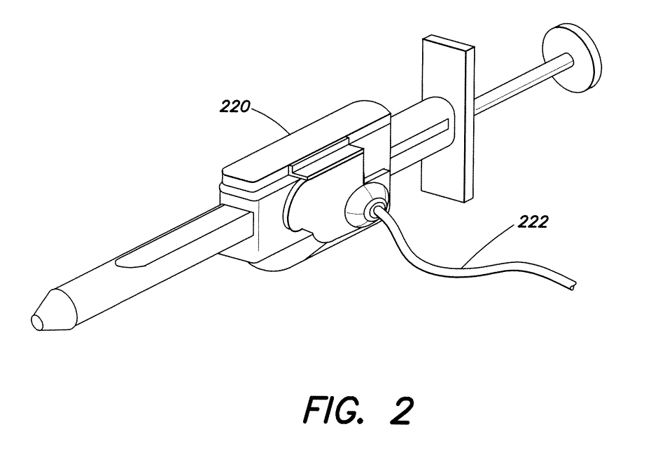

The vibration source is configured and arranged to vibrate at least one of the injector body and the plunger. The vibration source may be directly connected or otherwise operatively coupled to at least one of the injector body and the plunger. As shown in FIG. 2, vibration source 220 may be connected to the exterior of the injector or an interior surface. A power source for the vibration source may be attached to the injector so as to be supported by the injector, as shown in FIG. 2, or may be located in an external apparatus (not shown) such as a phacoemulsification apparatus, so that the power is provided by the external apparatus by a wire 222. Vibration may, for example, be generated mechanically or by using piezoelectric.

The vibration may be of any suitable kind so as to require a reduced force to move the IOL along at least a portion of the lumen as compared to a similar injector system that does not provide vibration. The vibration may be at one or more frequencies. The vibration may be shaped sinusoidally or as a square wave or any other suitable shape. The frequency may be constant or delivered in bursts (i.e., on/off, or full amplitude and reduced amplitude). The vibration source may be configured and arranged to cause longitudinal, lateral or rotational vibration of an injector component. In some embodiments, the frequency of the vibration delivered to the injector is between 1 Hz and 30 kHz.

Selection of a vibratory signal to reduce the force needed to actuate the plunger can be determined empirically by comparing the force needed for delivery when vibration is applied and when it is not applied. It will be appreciated that the appropriate vibratory signal will be determined by the shape of the lumen and plunger, and by the materials from which they are constructed. It will also be appreciated that consideration of any deleterious effects of any vibration on eye tissue must be considered when selecting a vibratory signal.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An intraocular lens injector, comprising:
an injector body having a lumen wall defining a lumen;
a plunger configured and arranged to move an IOL through the lumen; and
a vibration source configured and arrange to vibrate at least one of the injector body and the plunger.

2. The injector of claim 1, wherein the lumen wall is sized and shaped to fold or compress the IOL as it moves through the lumen.

3. The injector of claim 1, wherein the plunger includes threading to facilitate longitudinally through the lumen.

4. The injector of claim 1, wherein the injector is motorized to achieve longitudinal motion of the plunger through the lumen.

5. The injector of claim 1, wherein the vibration source is directly connected to injector body.

6. The injector of claim 5, wherein the vibration source is connected to an exterior surface of the injector.

7. The injector of claim 1, wherein the power source for vibration source is attached to the injector so as to be supported by the injector.

8. The injector of claim 1, wherein the power for the vibration source is provided by an external apparatus.
